(19) United States
(12) Patent Application Publication
(10) Pub. No.: US 2019/0021611 A1
(43) Pub. Date: Jan. 24, 2019

(54) APPARATUS AND METHOD FOR MEASURING BLOOD PRESSURE

(71) Applicant: SAMSUNG ELECTRONICS CO., LTD., Suwon-si (KR)

(72) Inventors: Yong Joo KWON, Yongin-si (KR); Jae Min KANG, Seoul (KR); Youn Ho KIM, Hwaseong-si (KR); Seung Woo NOH, Seongnam-si (KR); Sang Yun PARK, Hwaseong-si (KR)

(73) Assignee: SAMSUNG ELECTRONICS CO., LTD., Suwon-si (KR)

(21) Appl. No.: 15/934,435

(22) Filed: Mar. 23, 2018

(30) Foreign Application Priority Data
Jul. 24, 2017 (KR) 10-2017-0093788

(51) Int. Cl. A61B 5/021; A61B 5/0225

(52) U.S. Cl. CPC A61B 5/02108; A61B 5/0013; A61B 5/0225

(57) ABSTRACT

A blood pressure measuring apparatus includes a sensor configured to acquire a user image of a user, and a processor configured to determine, based on the user image, relative position information of a blood pressure measuring point of the user, the relative position information including a distance between a reference point of the user and the blood pressure measuring point, and measure a blood pressure of the user by correcting an effect of a hydrostatic pressure on the blood pressure, based on the relative position information that is determined.

### CROSS-REFERENCE TO RELATED APPLICATION

This application claims priority from Korean Patent Application No. 10-2017-0093788, filed on Jul. 24, 2017, in the Korean Intellectual Property Office, the disclosure of which is incorporated herein by reference in its entirety.

### BACKGROUND

### 1. Field

Apparatuses and methods consistent with example embodiments relate to an apparatus and method for measuring blood pressure, and more particularly to technology for measuring blood pressure by correcting the effect of hydrostatic pressure on blood pressure.

### 2. Description of the Related Art

To minimize the effect of hydrostatic pressure when blood pressure is measured, a user's wrist angle is measured by using a tri-axial acceleration sensor, and the blood pressure is measured only when the user's wrist is at the same level as the heart.

In this case, however, by using only an angle of a body part at which the blood pressure is measured, a position at the same level as the heart is determined, such that as the number of joints between the heart and the body part is increased, the number of variables in determining the position is also increased. This leads to significant errors in determining the position of a body part that is at the same level as the heart.

Further, blood pressure may be measured only at a determined position, causing discomfort to a user.

### SUMMARY

According to an aspect of an example embodiment, there is provided a blood pressure measuring apparatus a sensor configured to acquire a user image of a user, and a processor configured to determine, based on the user image, relative position information of a blood pressure measuring point of the user, the relative position information including a distance between a reference point of the user and the blood pressure measuring point, and measure a blood pressure of the user by correcting an effect of a hydrostatic pressure on the blood pressure, based on the relative position information that is determined.

The processor may be further configured to determine the distance between the reference point and the blood pressure measuring point by comparing a reference image of the user with the user image.

The processor may be further configured to determine the distance between the reference point and the blood pressure measuring point by comparing any one or any combination of a size of same feature points of the reference image and the user image, a position of the same feature points, and a distance between the same feature points.

The sensor may be further configured to sense a tilt of the blood pressure measuring apparatus, and the processor may be further configured to determine a height between the blood pressure measuring point and the reference point, based on the distance between the reference point and the blood pressure measuring point and the tilt that is sensed.

The processor may be further configured to, based on the height that is determined, correct the effect of the hydrostatic pressure on the blood pressure, using a hydrostatic pressure effect correction model for correcting the effect of the hydrostatic pressure on the blood pressure.

The sensor may include any one or any combination of a blood pressure measuring sensor, a tilt sensor, and a camera.

The processor may be further configured to determine, based on the user image, a blood pressure measuring posture of the user, as the relative position information.

The processor may be further configured to determine the blood pressure measuring posture, based on a result of a comparison of a reference image of the user with the user image, and a tilt of the blood pressure measuring apparatus, and correct the blood pressure that is measured, based on the blood pressure measuring posture that is determined.

The processor may be further configured to generate a guide image to guide the user to change either one or both of a blood pressure measuring posture of the user and a position of the blood pressure measuring apparatus.

The processor may be further configured to, in response to the blood pressure measuring posture being changed to a predetermined blood pressure measuring posture or the position of the blood pressure measuring apparatus being changed to a predetermined position, generate a reference image of the user, based on the user image.

The apparatus may further include an output interface configured to display any one or any combination of the user image, the guide image, the blood pressure that is measured, the hydrostatic pressure that is estimated, and the blood pressure that is corrected.

According to an aspect of an example embodiment, there is provided a blood pressure measuring method being performed by a blood pressure measuring apparatus, the method including acquiring a user image of a user, determining, based on the user image, relative position information of a blood pressure measuring point of the user, the relative position information including a distance between a reference point of the user and the blood pressure measuring point, and measuring a blood pressure of the user by correcting an effect of a hydrostatic pressure on the blood pressure, based on the relative position information that is determined.

The determining of the relative position information may include determining the distance between the reference point and the blood pressure measuring point by comparing a reference image of the user with the user image.

The determining of the distance between the reference point and the blood pressure measuring point may include determining the distance between the reference point and the blood pressure measuring point by comparing any one or any combination of a size of same feature points of the reference image and the user image, a position of the same feature points, and a distance between the same feature points.

The method may further include sensing a tilt of the blood pressure measuring apparatus, and the determining of the relative position information may include determining a height between the blood pressure measuring point and the reference point, based on the distance between the reference point and the blood pressure measuring point and the tilt that is sensed.

The measuring of the blood pressure may include, based on the height that is determined, correcting the effect of the hydrostatic pressure on the blood pressure, using a hydrostatic pressure effect correction model for correcting the effect of the hydrostatic pressure on the blood pressure.

The determining of the relative position information may include determining, based on the user image, a blood pressure measuring posture of the user, as the relative position information.

The determining of the relative position information may further include determining the blood pressure measuring posture, based on a result of a comparison of a reference image of the user with the user image, and a tilt of the blood pressure measuring apparatus, and the measuring of the blood pressure may include correcting the blood pressure that is measured, based on the blood pressure measuring posture that is determined.

The method may further include generating a guide image to guide the user to change either one or both of a blood pressure measuring posture of the user and a position of the blood pressure measuring apparatus, and in response to the blood pressure measuring posture being changed to a predetermined blood pressure measuring posture or the position of the blood pressure measuring apparatus being changed to a predetermined position, generating a reference image of the user, based on the user image.

The method may further include displaying any one or any combination of the user image, the guide image, the blood pressure that is measured, the hydrostatic pressure that is estimated, and the blood pressure that is corrected.

### BRIEF DESCRIPTION OF THE DRAWINGS

FIG. 1 is a block diagram illustrating a blood pressure measuring apparatus according to an example embodiment.

FIG. 2 is a diagram explaining a change in user images, according to a relative position change of a blood pressure measuring point with respect to a reference point, according to an example embodiment.

FIG. 3A is a diagram explaining user images, according to a change in a height difference between a reference point and a blood pressure measuring point, according to an example embodiment.

FIG. 3B is a diagram illustrating an image of a tilt change of a blood pressure measuring apparatus, according to an example embodiment.

FIG. 3C is a diagram illustrating user images, according to a change in a distance between a reference point and a blood pressure measuring point, according to an example embodiment.

FIG. 3D is a diagram explaining an example of determining a relative position of a blood pressure measuring point with respect to a reference point, according to an example embodiment.

FIG. 4 is a diagram explaining an example of generating a guide image and a reference image, according to an example embodiment.

FIG. 5 is a block diagram illustrating a blood pressure measuring apparatus according to another example embodiment.

FIG. 6 is a flowchart illustrating a blood pressure measuring method according to an example embodiment.

FIG. 7 is a flowchart illustrating a blood pressure measuring method according to another example embodiment.

FIG. 8 is a flowchart illustrating a blood pressure measuring method according to another example embodiment.

Throughout the drawings and the detailed description, unless otherwise described, the same drawing reference numerals will be understood to refer to the same elements, features, and structures. The relative size and depiction of these elements may be exaggerated for clarity, illustration, and convenience.

### DETAILED DESCRIPTION

Hereinafter, example embodiments of the present disclosure will be described in detail with reference to the accompanying drawings. In the drawings, the same reference symbols refer to same parts although illustrated in other drawings. In the following description, a detailed description of known functions and configurations incorporated herein will be omitted when it may obscure the subject matter of the example embodiments.

Process steps described herein may be performed differently from a specified order, unless the specified order is clearly stated in the context of the disclosure. That is, each step may be performed in a specified order, at substantially the same time, or in a reverse order.

Further, the terms used throughout this specification are defined in consideration of the functions according to the example embodiments, and can be varied according to a purpose of a user or manager, or precedent and so on. Therefore, definitions of the terms may be made on the basis of the overall context.

Any references to singular may include plural unless expressly stated otherwise. In the present specification, the terms, such as ‘including’ or ‘having,’ etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

In addition, the terms such as “unit,” “-er (-or),” and “module” described in the specification refer to an element for performing at least one function or operation, and may be implemented in hardware, software, or the combination of hardware and software.

Hereinafter, the example embodiments of an apparatus and method for measuring blood pressure will be described below with the accompanying drawings.

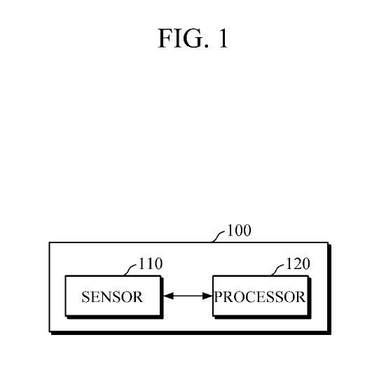

FIG. 1 is a block diagram illustrating a blood pressure measuring apparatus 100 according to an example embodiment.

Referring to FIG. 1, the blood pressure measuring apparatus 100 may correct hydrostatic pressure of the measured blood pressure by estimating a relative position of a blood pressure measuring point with respect to a reference point, and calculating hydrostatic pressure at the estimated relative position of the blood pressure measuring point, thereby minimizing the effect of hydrostatic pressure on blood pressure.

Here, the reference point is a body position of a blood pressure measuring target, and may be a position to be used a reference for determining a relative position of a blood pressure measuring point. Further, the blood pressure measuring point refers to a position of a blood pressure measuring point of a blood pressure measuring target, of which blood pressure is measured by using the blood pressure measuring apparatus 100. For example, the blood pressure measuring point may be a position of the blood pressure measuring apparatus 100.

For convenience of explanation, description below will be made based on an example embodiment in which the position of the blood pressure measuring apparatus 100 is used as the blood pressure measuring point, and the position of the heart as the reference point, the blood pressure measuring apparatus 100 may minimize the effect of hydrostatic pressure on blood pressure.

The blood pressure measuring apparatus 100 may be implemented as a software module or may be manufactured in the form of a hardware chip to be embedded in various types of electronic apparatuses. Examples of the electronic apparatuses may include a cellular phone, a smartphone, a tablet PC, a laptop computer, a personal digital assistant (PDA), a portable multimedia player (PMP), a navigation, an MP3 player, a digital camera, a wearable device, and the like, and examples of the wearable device may include a watch-type device, wristband-type device, a ring-type device, a waist belt-type device, a necklace-type device, an ankle band-type device, a thigh band-type device, a forearm band-type device, and the like. However, the electronic device is not limited to the above examples, and the wearable device is neither limited thereto.

Referring to FIG. 1, the blood pressure measuring apparatus 100 includes a sensor 110 and a processor 120. Here, the processor 120 may include one or more processors, a memory, and a combination thereof.

In an example embodiment, the sensor 110 may include a blood pressure measuring sensor to sense blood pressure of a user or a blood pressure measuring target, and one or more sensors to estimate a relative position of a blood pressure measuring point.

For example, the sensor 110 may include an image sensor or a camera to acquire a user's image. Here, the image sensor or the camera may include a Charge Coupled Device (CCD), a Complementary Metal Oxide Semiconductor (CMOS), and an electric image sensor, and examples of a camera include a depth camera and a 3-dimensional camera that may acquire distance or depth information and 2-dimensional pixel information.

The sensor 110 may include a position sensor (e.g., tilt sensor, acceleration sensor, gyro sensor, etc.) to sense a tilt, a motion, and a relative position of the blood pressure measuring apparatus 100, and may sense either one or both of a user image and a tilt of the blood pressure measuring apparatus 100.

The processor 120 may measure blood pressure of a user by extracting a relative position of a blood pressure measuring point with respect to a reference point, and by correcting the effect of hydrostatic pressure on blood pressure based on the extracted relative position of the blood pressure measuring point. To this end, the processor 120 may extract the relative position information of the blood pressure measuring point with respect to a reference point of a user based on the user image acquired from the sensor 110. Further, the processor 120 is not limited thereto, and may extract position information between blood pressure measuring points by using a tilt of the blood pressure measuring apparatus 100 and a user image that are acquired from the sensor 110.

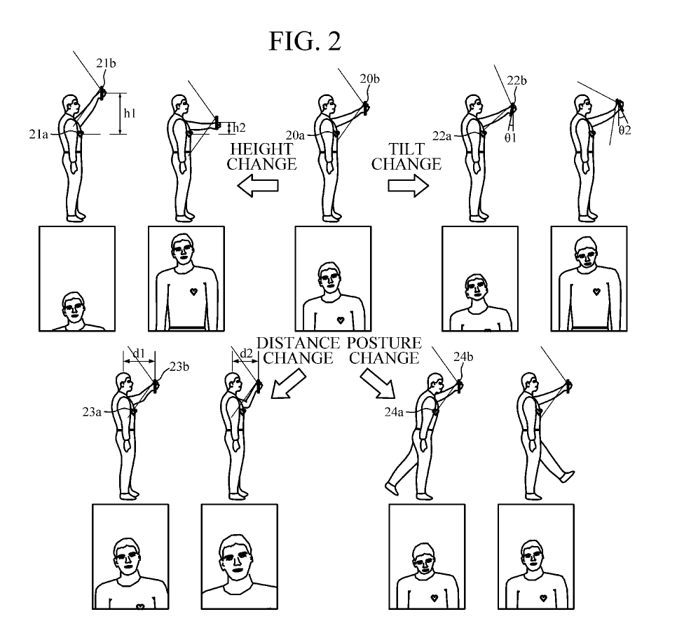

FIG. 2 is a diagram explaining a change in user images, according to a relative position change of a blood pressure measuring point with respect to a reference point, according to an example embodiment.

Referring to FIGS. 1 and 2, the processor 120 may use, as a reference image, user images captured at a predetermined reference point 20a and a predetermined blood pressure measuring point 20b with respect to the reference point.

Here, the reference image is an image used as a reference for determining a relative position of the blood pressure measuring point with respect to the reference point, and may be a user image of a predetermined blood pressure measuring posture or a predetermined position of the blood pressure measuring apparatus 100 acquired by the processor 120.

In an example embodiment, by comparing the reference image with the user image, the processor 120 may extract relative position information of the blood pressure measuring point, including the height, tilt, and distance of the blood pressure measuring point with respect to the reference point, and information on a user's blood pressure measuring posture.

For example, the processor 120 may estimate height differences h₁ and h₂ between a reference point 21a and a blood pressure measuring point 21b by comparing the reference point 20a of the reference image with the reference point 21a of a user image acquired by the sensor 110 when measuring blood pressure; and may determine whether the acquired user image is an image captured at a position higher or lower than a capturing height of the reference image. Further, based on the determination on the height of the user image, the processor 120 may estimate whether the height of the blood pressure measuring point 21b is higher or lower than the height of the blood pressure measuring point of the reference image, and may extract a relative position of the blood pressure measuring point 21b with respect to the reference point 21a. For example, upon determining that the user image is captured at a position higher than a capturing height of the reference image, the processor 120 may estimate that the blood pressure measuring point 21b is at a relatively higher position than the reference point 21a, and may extract relative position information of the blood pressure measuring point.

In another example, the processor 120 may estimate a relative position of a blood pressure measuring point 22b with respect to a reference point 22a by comparing the reference image with the user image acquired by the sensor 110 when measuring blood pressure, and by further using tilts θ₁ and θ₂ of the blood pressure measuring apparatus 100 that are sensed by the sensor 110. For example, the processor 120 may extract relative position information of the blood pressure measuring point in such a manner that upon analyzing that the position of the blood pressure measuring point 22b is higher or lower than the position of the blood pressure measuring point of the reference image based on comparison of the user image with the reference image, the tilt of the blood pressure measuring apparatus 100 is sensed by the sensor 110, and the processor 120 determines that an actual height of the blood pressure measuring point 22b is not changed.

In yet another example, the processor 120 may extract relative position information of a blood pressure measuring point 23b with respect to a reference point 23a in such a manner that by comparing the reference image with the user image acquired by the sensor 110 when measuring blood pressure, the processor 120 estimates whether the acquired user image is captured at a distance shorter or longer than a capturing distance of the reference image; and based on the determination, the processor 120 estimates whether distances d₁ and d₂ between the reference point 23a and the blood pressure measuring point 23b are shorter than a distance between the reference point and the blood pressure measuring point of the reference image. For example, upon determining that the user image is captured at a distance shorter than the capturing distance of the reference image, the processor 120 may extract relative position information of the blood pressure measuring point by estimating that the blood pressure measuring point is at a position relatively close to the reference point.

In yet another example, the processor 120 may sense a change in a user's blood pressure measuring posture based on comparison of the user image with the reference image and by using a tilt of the blood pressure measuring apparatus 100; and based on the sensed change in the blood pressure measuring posture, the processor 120 may extract relative position information of a blood pressure measuring point 24b with respect to a reference point 24a. For example, the processor 120 may extract relative position information of the blood pressure measuring point in such a manner that upon analyzing that the position of the blood pressure measuring point 24b of the user image is higher or lower than, or closer or further than, the blood pressure measuring point of the reference image based on an image analysis result of the user image and the reference image, if there is no change in the tilt sensed by the sensor 110, the processor 120 determines that tilts φ₁ and φ₂ of a user's body are changed; and based on the determination, the processor 120 determines that the user's posture is changed.

As described above, by estimating the height, tilt, and distance of the blood pressure measuring point with respect to the reference point, and the user's posture change based on the analysis of the user image, the processor 120 may extract relative position information of the blood pressure measuring point with respect to the reference point; and based on the tilt information sensed by the sensor 110, the processor 120 may estimate a relative position of the blood pressure measuring point more accurately.

Further, for convenience of explanation, examples of estimating the height, tilt, and distance of the blood pressure measuring point with respect to the reference point, and the user's posture change are described as separate example embodiments. However, when a user measures blood pressure by using the blood pressure measuring apparatus 100, a combination of two or more of the height, tilt, and distance of the blood pressure measuring point, and the user's posture change may be used; and even in this case, a relative position of the blood pressure measuring point with respect to the reference point may be estimated based on a combination of the above-described examples of extracting the relative position of the blood pressure measuring point.

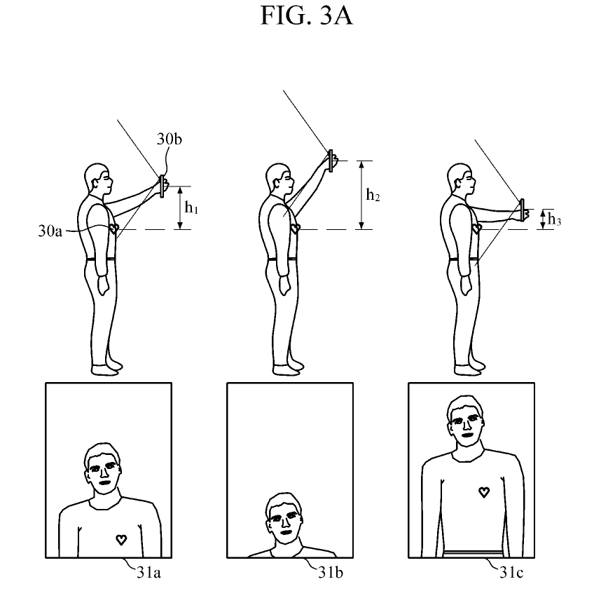

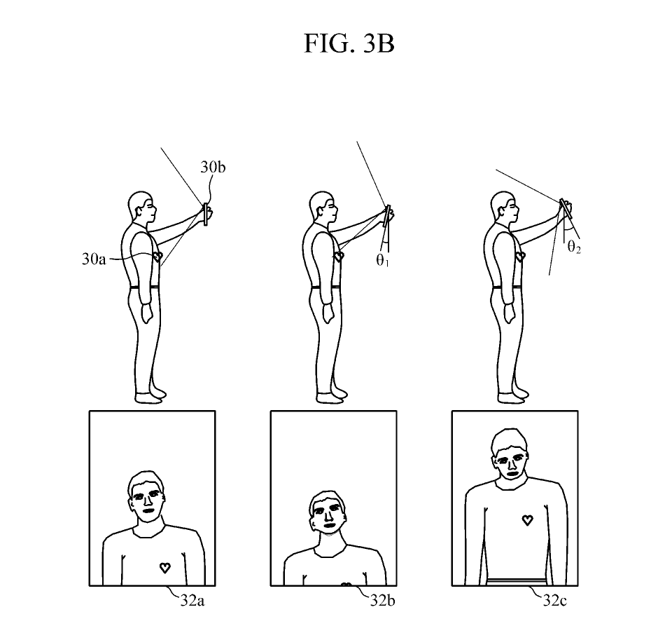

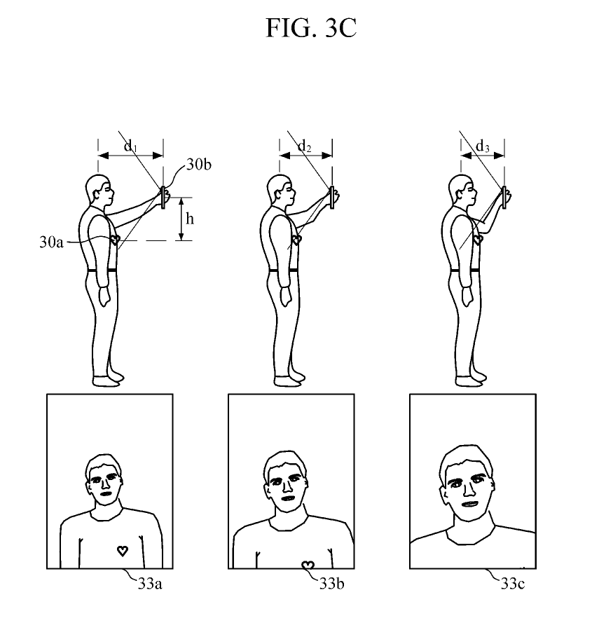

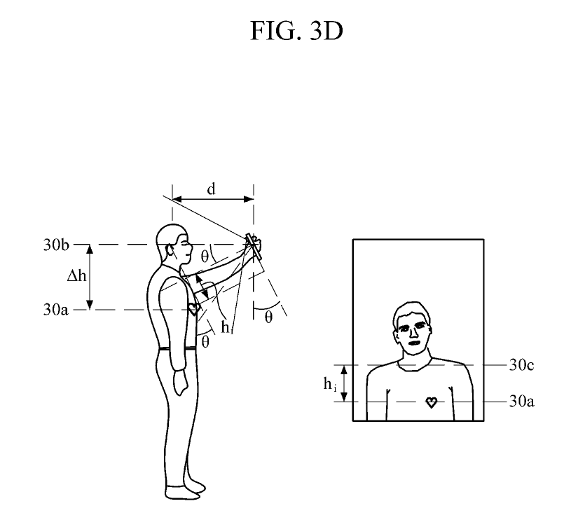

Hereinafter, a method of estimating, by the processor 120, a relative position of a blood pressure measuring point with respect to a reference point based on the analysis of a user image and a tilt change of the blood pressure measuring apparatus 100 will be described in detail with reference to FIGS. 3A to 3D.

FIG. 3A is a diagram illustrating user images 31a, 31b, and 31c, according to a change in a height difference between a reference point 30a and a blood pressure measuring point 30b, according to an example embodiment.

Referring to FIG. 3A, a user's figure in the user images 31a, 31b, and 31c acquired by the sensor 110 is changed according to a change in the height of the blood pressure measuring point 30b with respect to the reference point 30a. In this case, the processor 120 may estimate the height of the blood pressure measuring point with respect to the reference point by analyzing the user's figure in the user images 31a, 31b, and 31c.

In an example embodiment, the processor 120 may extract feature points from the user image and the reference image, and may estimate the height of the blood pressure measuring point based on the size and position of the feature points extracted from each image and the distance between the feature points.

Here, the feature points may refer to points that distinguish a user's face position, head direction, face, and torso. For example, the processor 120 may extract, as the feature point, at least one point of a user's eyes, nose, mouth, both ears, tip of the chin, and both shoulder points. However, the feature point is not limited thereto, and the processor 120 may extract a user's silhouette in the user image, and may use the extracted user's silhouette along with or instead of the feature points to compare images.

The processor 120 may calculate height differences h₁, h₂, and h₃ between the blood pressure measuring point 30a and the reference point 30b by extracting the feature points from the user image and the reference image, and by comparing the size and position of the feature points of the user image and corresponding feature points of the reference image.

For example, by comparing the feature points of the reference image with the feature points of the user image, the processor 120 may extract a position of the reference point in the user image; and by comparing the position of the reference point in the extracted user image with the position of the reference point in the reference image, the processor 120 may estimate a relative height of the blood pressure measuring point with respect to the reference point.

FIG. 3B is a diagram illustrating an image of a tilt change of a blood pressure measuring apparatus, according to an example embodiment.

Referring to FIG. 3B, with the height of the blood pressure measuring point with respect to the reference point being equal, user figures in user images 32a, 32b, and 32c acquired by the sensor 110 are changed according to the tilts θ₁ and θ₂ of the blood pressure apparatus 100. In this case, by only analyzing the user figures in the user images 32a, 32b, and 32c, the processor 120 may estimate a degree of tilt of the blood pressure measuring apparatus 100, and may extract a relative position of the blood pressure measuring point.

By comparing the position of the feature point in the reference image with the position of the feature point in the user image, the processor 120 may estimate the position of the reference point in the user image; and by comparing the estimated position of the reference point in the user image with the position of the reference point in the reference image, the processor 120 may estimate a change of height of the blood pressure measuring apparatus 100. In this case, by considering a tilt of the blood pressure measuring apparatus 100, the processor 120 may estimate that the blood pressure measuring apparatus 100 is simply inclined while being at the same height.

FIG. 3C is a diagram illustrating user images 33a, 33b, and 33c, according to a change in a distance between the reference point 30b and the blood pressure measuring point 30a, according to an example embodiment.

Referring to FIG. 3C, the user images 33a, 33b, and 33c acquired by the sensor 110 are changed according to distance differences d₁, d₂, and d₃ (e.g., difference between a direction of gravity and a vertical distance). In this case, based on the user images 33a, 33b, and 33c acquired by the sensor 110, the processor 120 may calculate differences of the distance between the blood pressure measuring point 30a and the reference point 30b.

For example, by comparing a distance between feature points of the user image with a distance between corresponding feature points of the reference image, the processor 120 may calculate differences of the distance between the blood pressure measuring point 30a and the reference point 30b. For example, in the case in which the distance between the feature points of the user image becomes larger than the distance between the feature points of the reference image, the processor 120 may determine that the distance between the reference point and the blood pressure measuring point in the user image is shorter than the distance between the reference point and the blood pressure measuring point in the reference image.

In another example, in the case in which a detected feature point of the user image is larger in size than the size of a corresponding feature point of the reference image, the processor 120 may determine that the user image is captured at a distance closer to a user than the reference image.

In yet another example, by analyzing the user image and sensing the tilt of the blood pressure measuring apparatus 100, the processor 120 may detect a change of a user's posture, and may determine a relative position of the blood pressure measuring point with respect to the reference point. For example, while the tilt of the blood pressure measuring apparatus 100 is the same as the tilt of the blood pressure measuring apparatus 100 when capturing the reference image, in the case in which a distance difference is detected in the user images, the processor 120 estimates that the tilt of a user's body is changed, and may further calculate information on a blood pressure measuring posture of the user.

In this case, the changed tilt of the user's body may be determined by comparing the feature points of the reference image and the feature points of the user images, and may be calculated by using the user images including depth information captured by using a distance measuring sensor or a depth camera.

For convenience of explanation, although examples of estimating the height and distance change of the blood pressure measuring point with respect to the reference point, a tilt change of the blood pressure measuring apparatus, and a user's posture change, are described as separate example embodiments, the processor 120 may estimate an accurate position of the blood pressure measuring point with respect to the reference point based on a combination of the above-described separate example embodiments.

FIG. 3D is a diagram explaining an example of extracting a relative position of the blood pressure measuring point 30a with respect to the reference point 30b, according to an example embodiment.

Referring to FIGS. 1 to 3D, based on the user image and the tilt of the blood pressure measuring apparatus, the processor may 120 extract a relative position of the blood pressure measuring point with respect to the reference point.

For example, by analyzing the user image, the processor 120 may calculate a distance hᵢ between a central line 30c of the user image and the reference point 30a in the user image. Here, the central line 30c is a virtual straight line that vertically divides the user image into two parts or may be a point of intersection of two virtual straight lines formed by connecting vertices of the user image. However, the central line 30c is not limited thereto, and a predetermine point in the user image may be used as a central point.

The processor 120 may calculate a distance d between the reference point 30a and the blood pressure measuring point 30b. For example, by comparing the distance between the feature points of the user image with the distance between the corresponding feature points of the reference image, the processor 120 may calculate differences of the distance between the blood pressure measuring point 30a and the reference point 30b. However, calculation of the distance is not limited thereto, and the processor 120 may directly acquire the distance d between the reference point and the blood pressure measuring point based on depth information and 2-dimensional pixel information obtained by using a depth camera and a 3-dimensional camera.

The processor 120 may estimate the position of the blood pressure measuring point 30b with respect to the reference point 30a based on the distance hᵢ between the central line of the user image and the reference point 30a in the user image, the distance d between the reference point 30a and the blood pressure measuring point 30b, and a tilt θ of the blood pressure measuring apparatus 100 that is obtained by the sensor 110. For example, upon calculating the distance d between the reference point 30a and the blood pressure measuring point 30b, and the tilt θ of the blood pressure measuring apparatus 100 that is obtained by the sensor 110, the processor 120 may calculate a height difference Δh between the blood pressure measuring point 30b and the reference point 30a by using a trigonometric function; and by using, as a correction value, the distance hᵢ between the central line 30c of the user image and the reference point 30a in the user image, the processor 120 may calculate an accurate height difference Δh between the blood pressure measuring point 30b and the reference point 30a.

However, this is an example, and the processor 120 may calculate a relative position of the blood pressure measuring point with respect to the reference point and the height difference Δh therebetween by using a geometrical modeling method, a known mathematical estimation method, and a position estimation model that is pre-generated based on the distance hᵢ between the central line 30c of the user image and the reference point in the user image, the distance d between the reference point and the blood pressure measuring point, and the tilt θ of the blood pressure measuring apparatus 100. In this case, the estimation model may be generated by machine learning.

In an example embodiment, the processor 120 may correct the effect of hydrostatic pressure on blood pressure based on information of a relative position of the blood pressure measuring point with respect to the reference point.

Blood pressure is affected by hydrostatic pressure depending on a measurement position of blood pressure. The hydrostatic pressure occurring in this case may vary depending on a relative position of the blood pressure measuring point with respect to the reference point. For example, in the case in which the reference point is the position of the heart, and the height of the reference point from the ground surface is the same as the height of the blood pressure measuring point from the ground surface, the effect of hydrostatic pressure on blood pressure may be minimized. However, in the case in which there is a difference in height between the reference point and the blood pressure measuring point due to a change of relative position of the blood pressure point, blood pressure measured at the blood pressure measuring point may be different, due to the effect of hydrostatic pressure, from blood pressure measured when the height of the reference point from the ground surface is the same as the height of the blood pressure measuring point from the ground surface.

Further, the effect of hydrostatic pressure on the measured blood pressure may vary depending on a user's blood pressure measuring posture, e.g., a supine posture or a sitting posture. Based on relative position information of the blood pressure measuring point with respect to the reference point and a user's blood pressure measuring posture, the processor 120 may correct the effect of hydrostatic pressure on blood pressure.

For example, upon determining the relative position of the blood pressure measuring point with respect to the reference point, the processor 120 may calculate a difference of height from the ground surface between the reference point and the blood pressure measuring point by using a geometrical modeling method and a known mathematical estimation method. In this case, a method of calculating the difference of height from the ground surface therebetween is described above, such that overlapping description thereof will be omitted.

Upon extracting height information of the reference point and the blood pressure measuring point, the processor 120 may correct the effect of hydrostatic pressure on blood pressure of a user by using a hydrostatic pressure effect correction model for correcting a hydrostatic pressure effect on blood pressure.

Here, the hydrostatic pressure effect correction model may be a model including a hydrostatic pressure correction value according to relative position information of the blood pressure measuring point with respect to the reference point. For example, the hydrostatic pressure effect correction model may be a correction model generated by mathematically and experimentally calculating a hydrostatic pressure correction value according to the heights, from the ground surface, of the reference point and the blood pressure measuring point and a difference between the heights, a hydrostatic pressure correction value according to a straight line between the reference point and the blood pressure measuring point, and a hydrostatic pressure correction value according to a user's blood pressure measuring posture.

Further, hydrostatic pressure may also be corrected by using a known hydrostatic pressure according to a height difference between the reference point and the blood pressure measuring point; but the processor 120 may use a hydrostatic pressure effect correction model generated based on learning data including information on a change of hydrostatic pressure measured at a relative position of the blood pressure measuring point with respect to the reference point in a 3-dimensional space.

For example, upon calculating heights of the reference point and the blood pressure measuring point, in the case in which the height of the blood pressure measuring point is higher than the height of the reference point, the measured blood pressure may be lower, due to the effect of hydrostatic pressure, than blood pressure measured when the height of the blood pressure measuring point is the same as the height of the reference point. In this case, the processor 120 may calculate a corrected blood pressure by adding a hydrostatic pressure correction value to the measured blood pressure.

Further, upon calculating heights of the reference point and the blood pressure measuring point, in the case in which the height of the blood pressure measuring point is lower than the height of the reference point, the measured blood pressure may be higher, due to the effect of hydrostatic pressure, than blood pressure measured when the height of the blood pressure measuring point is the same as the height of the reference point. In this case, the processor 120 may calculate a corrected blood pressure by adding a hydrostatic pressure correction value to the measured blood pressure. Here, the added hydrostatic pressure correction value may be a positive value or a negative value according to a height difference between the reference point and the blood pressure measuring point.

hydrostatic pressure (p)=μgh [Equation 1]

For example, Equation 1 is an equation to calculate hydrostatic pressure (p) without considering other factors, wherein ρ denotes the density of blood, g denotes acceleration of gravity, and h denotes the depth of liquid.

In this case, referring to FIG. 3D and Equation 1, the hydrostatic pressure (p₃₀ₐ) of the reference point and the hydrostatic pressure (p_{30b}) of the blood pressure measuring point are represented as (p₃₀ₐ)=ρgh₃₀ₐ and (p_{30b})=ρgh_{30b}, respectively.

Here, the difference of hydrostatic pressure due to the height difference between the reference point and the blood pressure measuring point may be determined to be p₃₀ₐ−p_{30b}=ρg(h₃₀ₐ−h_{30b}), in which case by using, as a reference height, the height of the reference point 30a with respect to a direction of gravity, the hydrostatic pressure resulting from the height difference between the reference point 30a and the blood pressure measuring point 30b has a negative value. Accordingly, in the case in which the reference point is the position of heart, and the blood pressure measuring point is at a higher position than the position of heart, the measured blood pressure is lower, due to the effect of hydrostatic pressure, than the blood pressure measured when the height of the blood pressure measuring point is the same as the height of the reference point. In this case, considering the effect of hydrostatic pressure, the processor 120 may correct the measured blood pressure by adjusting the measured blood pressure to a higher level.

In this manner, by correcting hydrostatic pressure of the measured blood pressure according to the height difference between the reference point 30a and the blood pressure measuring point 30b, the processor 120 may measure an accurate blood pressure value even when there is a relative position difference between the reference point and the blood pressure measuring point.

In another example, the processor 120 may calculate blood pressure that is corrected according to a user's blood pressure measuring posture. For example, when the user's blood pressure measuring posture is a posture, such as a supine or prone posture, in which pressure is applied to the heart or the blood pressure measuring point, the processor 120 may correct the measured blood pressure by adjusting the measured blood pressure of the user to a lower level based on the estimated blood pressure measuring posture.

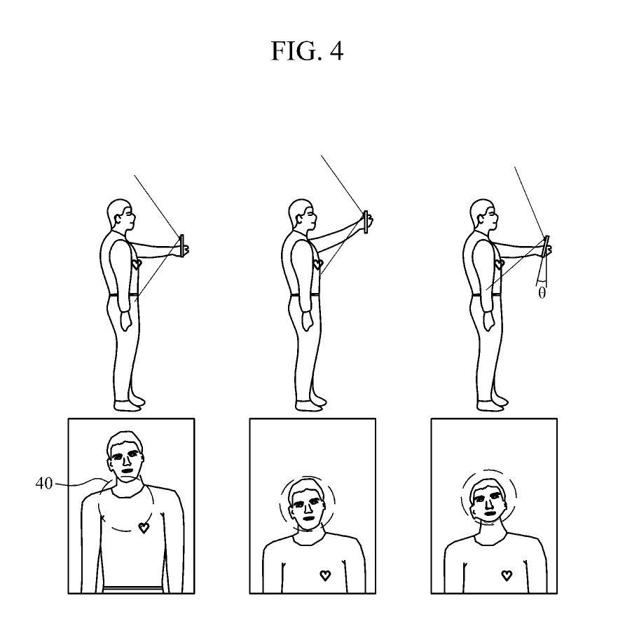

FIG. 4 is a diagram explaining an example of generating a guide image 40 and a reference image, according to an example embodiment.

Referring to FIG. 4, the processor 120 may generate the guide image 40 to change either one or both of the blood pressure measuring posture and the position of the blood pressure measuring apparatus. The processor 120 may generate the guide image 40 to induce a user to take a predetermined blood pressure measuring posture or to induce the blood pressure measuring apparatus to move to a predetermined position, to measure blood pressure accurately.

For example, the processor 120 may generate the guide image 40, and may determine whether a user's face is included in the guide image 40. In the case in which the user's face is not included in the guide image 40, the processor 120 may induce a user to change a blood pressure measuring posture so that blood pressure of the user may be measured according to a predetermined posture. Further, the processor 120 may generate a visual alarm (e.g., color change of the guide image, guidance message, etc.), an audible alarm (e.g., beep sound, etc.), and a tactile alarm (e.g., vibration, etc.), to induce the user to take a predetermined posture according to the guide image 40.

In addition, the processor 120 may extract feature points not only from a user's face but also from the acquired user image, and may generate the guide image to induce a position and size of each feature point and a distance between the feature points to be included in a predetermined range.

In another example, in the case in which the tilt θ of the blood pressure measuring apparatus 100, which is obtained from the sensor 110, is not within a predetermined range, the processor 120 may generate an alarm to move the blood pressure measuring apparatus 100, to induce the blood pressure measuring apparatus 100 to measure blood pressure at a predetermined blood pressure measuring point.

Further, in the case in which, according to the guide image, a user takes a predetermined blood pressure measuring posture or the blood pressure measuring apparatus is placed at a predetermined position, the processor 120 may acquire a user image to generate a reference image.

For example, at the time of initialization of the blood pressure apparatus 100 or in the initial attempt to measure blood pressure, the processor 120 may generate and output the guide image to induce a user to take a predetermined posture, or may generate an alarm to induce the user to move the blood pressure measuring apparatus 100 to a posture or position according to the guide image; and in the case in which, according to the generated guide image or alarm, the user takes a predetermined posture or the blood pressure measuring apparatus 100 is placed at a position, the processor 120 may capture a user image to generate a reference image.

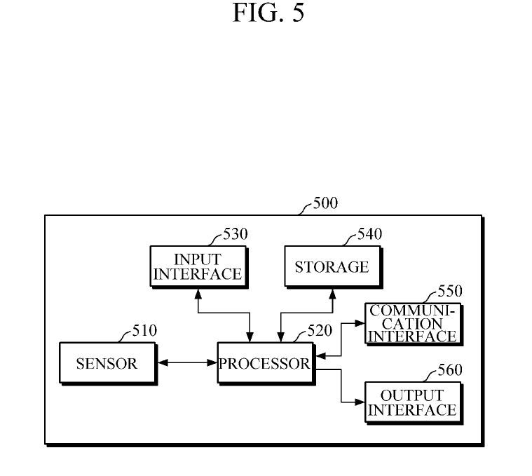

FIG. 5 is a block diagram illustrating a blood pressure measuring apparatus 500 according to another example embodiment.

Referring to FIG. 5, the blood pressure measuring apparatus 500 includes a sensor 510, a processor 520, an input interface 530, a storage 540, a communication interface 550, and an output interface 560. Here, the sensor 510 and the processor 520 may perform the same functions as the sensor 110 and the processor 120 illustrated with reference to FIG. 1, such that description below will be made based on details that do not overlap.

The input interface 530 may receive input of various operation signals from a user.

In an example embodiment, the input interface 530 may include a keypad, a dome switch, a touch pad (static pressure/capacitance), a jog wheel, a jog switch, a hardware (H/W) button, and the like. The touch pad, which forms a layer structure with a display, may be called a touch screen.

The storage 540 may store programs or commands for operation of a scattering coefficient measurement apparatus, and may store data input to and output from the blood pressure measuring apparatus 500. For example, the storage 540 may store intensity data measured by an optical detector array 540, a user's blood pressure calculated by the processor 520, and the like.

The storage 540 may include at least one storage medium of a flash memory type memory, a hard disk type memory, a multimedia card micro type memory, a card type memory (e.g., an SD memory, an XD memory, etc.), a Random Access Memory (RAM), a Static Random Access Memory (SRAM), a Read Only Memory (ROM), an Electrically Erasable Programmable Read Only Memory (EEPROM), a Programmable Read Only Memory (PROM), a magnetic memory, a magnetic disk, and an optical disk, and the like. Further, the blood pressure measuring apparatus 500 may operate an external storage medium, such as web storage and the like, which performs a storage function of the storage 540 on the Internet.

The communication interface 550 may perform communication with an external device. For example, the communication interface 550 may transmit, to the external device, data input from a user through the input interface 530, the user image acquired by the sensor 110, position information and a blood pressure measurement value of the blood pressure measuring apparatus 500, and the relative position information of the blood pressure measuring point with respect to the reference point, a hydrostatic pressure correction value, and the like, which are calculated by the processor 520; or may receive various data, such as a hydrostatic pressure correction model and the like, from the external device.

In this case, the external device may be medical equipment using information on the measured blood pressure and the corrected blood pressure, a printer to print out results, or a display to display the measured blood pressure and/or a hydrostatic pressure correction value, and the corrected blood pressure data. In addition, the external device may be a digital TV, a desktop computer, a cellular phone, a smartphone, a tablet PC, a laptop computer, a personal digital assistant (PDA), a portable multimedia player (PMP), a navigation, an MP3 player, a digital camera, a wearable device, and the like, but is not limited thereto.

The communication interface 550 may communicate with external devices by using Bluetooth communication, Bluetooth Low Energy (BLE) communication, Near Field Communication (NFC), WLAN communication, Zigbee communication, Infrared Data Association (IrDA) communication, Wi-Fi Direct (WFD) communication, Ultra Wideband (UWB) communication, Ant+ communication, WIFI communication, Radio Frequency Identification (RFID) communication, 3G communication, 4G communication, 5G communication, and the like. However, this is an example, and the communication part is not limited thereto.

The output interface 560 may display any one or any combination of a user image, a guide image, the measured blood pressure of a user, the estimated hydrostatic pressure, and the corrected blood pressure.

In an example embodiment, the output interface 560 may output any one or any combination of the user image, the guide image, the measured blood pressure of a user, the estimated hydrostatic pressure, and the corrected blood pressure by using any one or any combination of an acoustic method, a visual method, and a tactile method. To this end, the output interface 560 may include a display, a speaker, a vibrator, and the like.

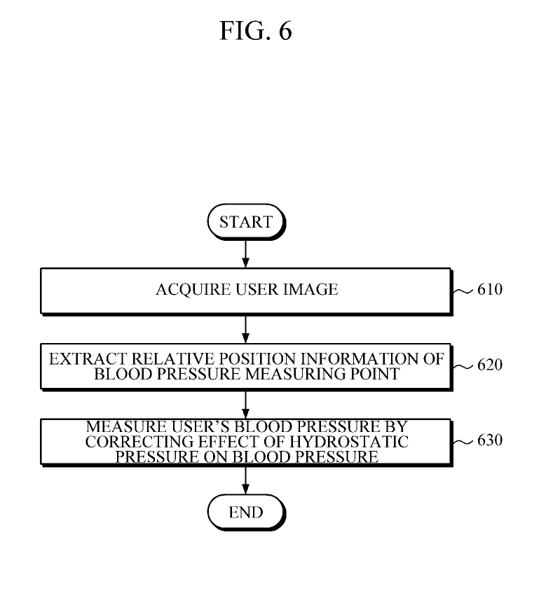

FIG. 6 is a flowchart illustrating a blood pressure measuring method, according to an example embodiment. The blood pressure measuring method of FIG. 6 may be performed by the blood pressure apparatuses 100 and 500 of FIGS. 1 and 5.

Referring to FIGS. 1 to 6, the blood pressure measuring apparatus 100 may acquire a user image in operation 610.

For example, the blood pressure measuring apparatus 100 may acquire the user image by using an image sensor or a camera to acquire the user image.

Based on the acquired user image, the blood pressure measuring apparatus 100 may extract relative position information of a blood pressure measuring point, which includes a distance between the reference point and the blood pressure measuring point of a user, in operation 620.

For example, the blood pressure measuring apparatus 100 may determine a relative position of the blood pressure measuring point with respect to the reference point, by comparing the reference image with the user image, and by estimating the height, tilt, and distance of the blood pressure measuring point with respect to the reference point, and a user's posture change.

The blood pressure measuring apparatus 100 may measure a user's blood pressure by correcting the effect of hydrostatic pressure on blood pressure based on the extracted relative position information of the blood pressure measuring point, in operation 630.

In an example embodiment, the blood pressure measuring apparatus 100 may correct the effect of hydrostatic pressure on a user's blood pressure by using a hydrostatic pressure effect correction model for correcting the hydrostatic pressure effect on blood pressure.

For example, upon determining the relative position of the blood pressure measuring point with respect to the reference point, the blood pressure measuring apparatus 100 may calculate a difference of height, from the ground surface, between the reference point and the blood pressure measuring point by using a geometrical modeling method and a known mathematical estimation method; and upon extracting height information of the reference point and the blood pressure measuring point, the blood pressure measuring apparatus 100 may measure blood pressure by correcting the effect of hydrostatic pressure on a user's blood pressure by using the hydrostatic pressure effect correction model.

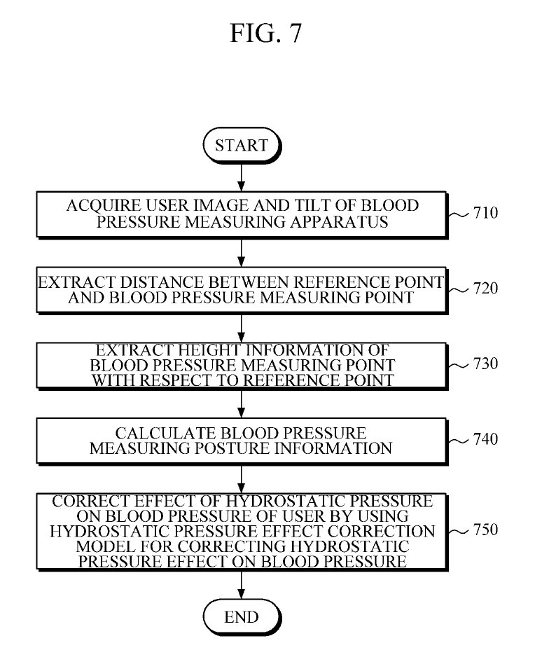

FIG. 7 is a flowchart illustrating a blood pressure measuring method, according to another example embodiment. The blood pressure measuring method of FIG. 7 may be performed by the blood pressure apparatuses 100 and 500 of FIGS. 1 and 5.

In an example embodiment, the blood pressure measuring apparatus 500 may include an image sensor or a camera to acquire a user image, and a position sensor (e.g., tilt sensor, acceleration sensor, gyro sensor, etc.) to sense a tilt, motion, and a relative position of the blood pressure measuring apparatus 500, and the sensors may acquire either one or both of the user image and the tilt of the blood pressure measuring apparatus 500, in operation 710.

The blood pressure measuring apparatus 500 may extract a distance between the reference point and the blood pressure measuring point by comparing the reference image and the user image, in operation 720. For example, the user image captured by the blood pressure measuring apparatus 500 may be changed according to a distance difference between the blood pressure measuring point and the reference point. In this case, based on the acquired user image, the blood pressure measuring apparatus 500 may calculate a distance difference between the blood pressure measuring point and the reference point.

In an example embodiment, by comparing the distance between the feature points of the user image with the distance between the corresponding feature points of the reference image, the blood pressure measuring apparatus 500 may calculate a distance difference between the blood pressure measuring point and the reference point.

For example, in the case in which the distance between the feature points of the user image becomes larger than the distance between the feature points of the reference image, the blood pressure measuring apparatus 500 may determine that the distance between the reference point and the blood pressure measuring point is shorter than the distance between the reference point and the blood pressure measuring point in the reference image.

Here, the feature points may refer to points that distinguish a user's face position, head direction, face, and torso. For example, the blood pressure measuring apparatus 500 may extract, as the feature point, at least one point of a user's eyes, nose, mouth, both ears, tip of the chin, and both shoulder points. However, the feature point is not limited thereto, and the blood pressure measuring apparatus 500 may extract a user's silhouette in the user image, and may use the extracted user's silhouette along with or instead of the feature points to compare images.

In another example, in the case in which a detected feature point of the user image is larger in size than the size of a corresponding feature point of the reference image, the blood pressure measuring apparatus 500 may determine that the user image is captured at a distance closer to a user than the reference image.

The blood pressure measuring apparatus 500 may extract height information of the blood pressure measuring point with respect to the reference point based on the distance between the reference point and the blood pressure measuring point, which is the relative position information, and based on information on the sensed tilt, in operation 730.

For example, it can be seen that a user's figure in user images acquired by the blood pressure measuring apparatus 500 is changed according to a change in the height of the blood pressure measuring point with respect to the reference point. Accordingly, the blood pressure measuring apparatus 500 may estimate the height of the blood pressure measuring point with respect to the reference point by analyzing the user's figure in the user images. For example, the blood pressure measuring apparatus 500 may estimate the height of the blood pressure measuring point with respect to the reference point based on the size and position of the feature points extracted from the user image and the reference image, and the distance between the feature points.

By analyzing the user image and sensing the tilt of the blood pressure measuring apparatus 500, the blood pressure measuring apparatus 500 may detect a change in a user's blood pressure measuring posture, and may determine a relative position of the blood pressure measuring point with respect to the reference point, i.e., calculate blood pressure measuring posture information, in operation 740. For example, while the tilt of the blood pressure measuring apparatus 500 is the same as the tilt of the blood pressure measuring apparatus 500 when capturing the reference image, in the case in which a distance difference is detected in the user images, the blood pressure measuring apparatus 500 estimates that the tilt of a user's body is changed, and may further calculate information on a blood pressure measuring posture of the user.

In this case, the changed tilt of the user's body may be determined by comparing the feature points of the reference image and the feature points of the user images, and may be calculated by using the user images including depth information captured by using a distance measuring sensor or a depth camera.

The blood pressure measuring apparatus 500 may correct the effect of hydrostatic pressure on blood pressure of a user by using a hydrostatic pressure effect correction model for correcting a hydrostatic pressure effect on blood pressure, in operation 750. For example, the blood pressure measuring apparatus 500 may correct the effect of hydrostatic pressure on blood pressure of a user by using a correction model for correcting the hydrostatic pressure effect on blood pressure based on the distance between the reference point and the blood pressure measuring point, which is relative position information, height information of the blood pressure measuring point with respect to the reference point, and the sensed tilt information of the blood pressure measuring apparatus 500.

In an example embodiment, the blood pressure measuring apparatus 500 may correct the effect of hydrostatic pressure on the measured blood pressure by using the hydrostatic pressure effect correction model, which is generated by mathematically and experimentally calculating a hydrostatic pressure correction value according to the heights, from the ground surface, of the reference point and the blood pressure measuring point and a difference between the heights, a hydrostatic pressure correction value according to a straight line between the reference point and the blood pressure measuring point, and a hydrostatic pressure correction value according to a user's blood pressure measuring posture.

For example, upon calculating heights of the reference point and the blood pressure measuring point, in the case in which the height of the blood pressure measuring point is higher than the height of the reference point, the measured blood pressure may be lower, due to the effect of hydrostatic pressure, than blood pressure measured when the height of the blood pressure measuring point is the same as the height of the reference point. In this case, the blood pressure measuring apparatus 500 may calculate a corrected blood pressure by adding a hydrostatic pressure correction value to the measured blood pressure.

Further, upon calculating heights of the reference point and the blood pressure measuring point, in the case in which the height of the blood pressure measuring point is lower than the height of the reference point, the measured blood pressure may be higher, due to the effect of hydrostatic pressure, than blood pressure measured when the height of the blood pressure measuring point is the same as the height of the reference point. In this case, the blood pressure measuring apparatus 500 may calculate a corrected blood pressure by adding a hydrostatic pressure correction value to the measured blood pressure. Here, the added hydrostatic pressure correction value may be a positive value or a negative value according to a height difference between the reference point and the blood pressure measuring point

In this manner, by correcting hydrostatic pressure of the measured blood pressure according to the height difference between the reference point and the blood pressure measuring point, the blood pressure measuring apparatus 500 may measure an accurate blood pressure value even when there is a relative position difference between the reference point and the blood pressure measuring point.

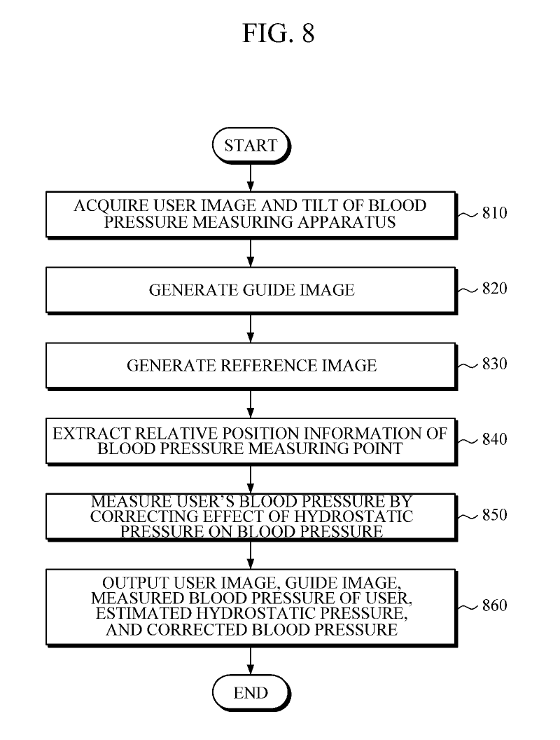

FIG. 8 is a flowchart illustrating a blood pressure measuring method, according to another example embodiment. The blood pressure measuring method of FIG. 8 may be performed by the blood pressure measuring apparatus 500 of FIG. 5.

In an example embodiment, the blood pressure measuring apparatus 500 may acquire either one or both of the user image and the tilt of the blood pressure measuring apparatus 500, in operation 810.

The blood pressure measuring apparatus 500 may generate a guide image to change either one or both of the blood pressure measuring posture and the position of the blood pressure measuring apparatus 500 based on either one or both of the sensed user image and the tilt of the blood pressure measuring apparatus 500, in operation 820.

For example, in the case in which a user's face position of the user image is not included in the guide image, the blood pressure measuring apparatus 500 may generate the guide image to induce the user to change a blood pressure measuring posture, so that the user's face may be included in the guide image, and blood pressure of the user may be measured according to a predetermined posture.

In another example, in the case in which a tilt of the blood pressure measuring apparatus 500 is not within a predetermined range, the blood pressure measuring apparatus 500 may generate an alarm to move the blood pressure measuring apparatus 500, so that blood pressure may be measured at a predetermined blood pressure measuring point.

Further, in the case in which, according to the guide image, a user takes a predetermined blood pressure measuring posture or the blood pressure measuring apparatus is placed at a predetermined position, the blood pressure measuring apparatus 500 may acquire a user image to generate a reference image, in operation 830. For example, at the time of initialization of the blood pressure apparatus 100 or in the initial attempt to measure blood pressure, the blood pressure measuring apparatus 500 may generate and output the guide image to induce a user to take a predetermined posture, or may generate an alarm to induce the user to move the blood pressure measuring apparatus 500 to a posture or position according to the guide image; and in the case in which, according to the generated guide image or alarm, the user takes a predetermined posture or the blood pressure measuring apparatus 500 is placed at a position, the blood pressure measuring apparatus 500 may capture a user image to generate a reference image.

Based on the acquired user image, the blood pressure measuring apparatus 500 may extract relative position information of the blood pressure measuring point, which includes the distance between the reference point and the blood pressure measuring point of the user, in operation 840.

The blood pressure measuring apparatus 500 may measure the user's blood pressure by correcting the effect of hydrostatic pressure on blood pressure based on the extracted relative position information of the blood pressure measuring point, in operation 850.

The blood pressure measuring apparatus 500 may output any one or any combination of the user image, the guide image, the measured blood pressure of the user, the estimated hydrostatic pressure, and the corrected blood pressure by using any one or any combination of an acoustic method, a visual method, and a tactile method, in operation 860.

The present disclosure can be realized as a computer-readable code written on a computer-readable recording medium. Codes and code segments for realizing the present disclosure can be easily deduced by computer programmers of ordinary skill in the art. The computer-readable recording medium may be any type of recording device in which data is stored in a computer-readable manner. Examples of the computer-readable recording medium include a ROM, a RAM, a CD-ROM, a magnetic tape, a floppy disc, an optical disk, and the like. Further, the computer-readable recording medium can be distributed over a plurality of computer systems connected to a network so that a computer-readable recording medium is written thereto and executed therefrom in a decentralized manner.

A number of examples have been described above. Nevertheless, various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A blood pressure measuring apparatus comprising:
a sensor configured to acquire a user image of a user; and
a processor configured to:
determine, based on the user image, relative position information of a blood pressure measuring point of the user, the relative position information comprising a distance between a reference point of the user and the blood pressure measuring point; and
measure a blood pressure of the user by correcting an effect of a hydrostatic pressure on the blood pressure, based on the relative position information that is determined.

2. The apparatus of claim 1, wherein the processor is further configured to determine the distance between the reference point and the blood pressure measuring point by comparing a reference image of the user with the user image.

3. The apparatus of claim 2, wherein the processor is further configured to determine the distance between the reference point and the blood pressure measuring point by comparing any one or any combination of a size of same feature points of the reference image and the user image, a position of the same feature points, and a distance between the same feature points.

4. The apparatus of claim 1, wherein the sensor is further configured to sense a tilt of the blood pressure measuring apparatus, and
the processor is further configured to determine a height between the blood pressure measuring point and the reference point, based on the distance between the reference point and the blood pressure measuring point and the tilt that is sensed.

5. The apparatus of claim 4, wherein the processor is further configured to, based on the height that is determined, correct the effect of the hydrostatic pressure on the blood pressure, using a hydrostatic pressure effect correction model for correcting the effect of the hydrostatic pressure on the blood pressure.

6. The apparatus of claim 1, wherein the sensor comprises any one or any combination of a blood pressure measuring sensor, a tilt sensor, and a camera.

7. The apparatus of claim 1, wherein the processor is further configured to determine, based on the user image, a blood pressure measuring posture of the user, as the relative position information.

8. The apparatus of claim 7, wherein the processor is further configured to:
determine the blood pressure measuring posture, based on a result of a comparison of a reference image of the user with the user image, and a tilt of the blood pressure measuring apparatus; and
correct the blood pressure that is measured, based on the blood pressure measuring posture that is determined.

9. The apparatus of claim 1, wherein the processor is further configured to generate a guide image to guide the user to change either one or both of a blood pressure measuring posture of the user and a position of the blood pressure measuring apparatus.

10. The apparatus of claim 9, wherein the processor is further configured to, in response to the blood pressure measuring posture being changed to a predetermined blood pressure measuring posture or the position of the blood pressure measuring apparatus being changed to a predetermined position, generate a reference image of the user, based on the user image.

11. The apparatus of claim 10, further comprising an output interface configured to display any one or any combination of the user image, the guide image, the blood pressure that is measured, the hydrostatic pressure that is estimated, and the blood pressure that is corrected.

12. A blood pressure measuring method being performed by a blood pressure measuring apparatus, the method comprising:
acquiring a user image of a user;
determining, based on the user image, relative position information of a blood pressure measuring point of the user, the relative position information comprising a distance between a reference point of the user and the blood pressure measuring point; and
measuring a blood pressure of the user by correcting an effect of a hydrostatic pressure on the blood pressure, based on the relative position information that is determined.

13. The method of claim 12, wherein the determining of the relative position information comprises determining the distance between the reference point and the blood pressure measuring point by comparing a reference image of the user with the user image.

14. The method of claim 13, wherein the determining of the distance between the reference point and the blood pressure measuring point comprises determining the distance between the reference point and the blood pressure measuring point by comparing any one or any combination of a size of same feature points of the reference image and the user image, a position of the same feature points, and a distance between the same feature points.

15. The method of claim 12, further comprising sensing a tilt of the blood pressure measuring apparatus,
wherein the determining of the relative position information comprises determining a height between the blood pressure measuring point and the reference point, based on the distance between the reference point and the blood pressure measuring point and the tilt that is sensed.

16. The method of claim 15, wherein the measuring of the blood pressure comprises, based on the height that is determined, correcting the effect of the hydrostatic pressure on the blood pressure, using a hydrostatic pressure effect correction model for correcting the effect of the hydrostatic pressure on the blood pressure.

17. The method of claim 12, wherein the determining of the relative position information comprises determining, based on the user image, a blood pressure measuring posture of the user, as the relative position information.

18. The method of claim 17, wherein the determining of the relative position information further comprises determining the blood pressure measuring posture, based on a result of a comparison of a reference image of the user with the user image, and a tilt of the blood pressure measuring apparatus; and
the measuring of the blood pressure comprises correcting the blood pressure that is measured, based on the blood pressure measuring posture that is determined.

19. The method of claim 12, further comprising:
generating a guide image to guide the user to change either one or both of a blood pressure measuring posture of the user and a position of the blood pressure measuring apparatus; and
in response to the blood pressure measuring posture being changed to a predetermined blood pressure measuring posture or the position of the blood pressure measuring apparatus being changed to a predetermined position, generating a reference image of the user, based on the user image.

20. The method of claim 19, further comprising displaying any one or any combination of the user image, the guide image, the blood pressure that is measured, the hydrostatic pressure that is estimated, and the blood pressure that is corrected.
